(19) United States
(12) Patent Application Publication
(10) Pub. No.: US 2026/0279577 A1
(43) Pub. Date: Sep. 17, 2026

(54) TECHNOLOGIES FOR MACHINE LEARNING CLOUD BASED CLINICAL OUTCOME PREDICTION

(71) Applicant: Care Predictor LLC, Dana Point, CA (US)

(72) Inventors: Loren Martin, Glendora, CA (US); Michael Krause, Irvine, CA (US); Michael Castanon, Dana Point, CA (US)

(21) Appl. No.: 19/668,010

(22) Filed: May 5, 2026

Related U.S. Application Data
(63) Continuation-in-part of application No. 18/350,663, filed on Jul. 11, 2023.
(60) Provisional application No. 63/368,439, filed on Jul. 14, 2022.

(51) Int. Cl. G16H 50/20; G06N 20/00

(52) U.S. Cl. CPC G16H 50/20; G06N 20/00

(57) ABSTRACT

Technologies for clinical outcome predictive analysis include a computing device that extracts provider features from an assessment index that are indicative of relational characteristics of a care provider. The computing device determines a clinical outcome performance metric associated with the care provider. The computing device links the provider features and the performance metric in training data and trains a prediction model to predict an outcome score based on provider feature. A computing device may predict a predicted outcome score based on extracted provider features using the trained prediction model and normalize the predicted outcome score to a predicted outcome index. The computing device may monitor ongoing therapeutic alliance status data and predict a risk of alliance failure using a trained prediction model. Other embodiments are described and claimed.

### CROSS-REFERENCE TO RELATED APPLICATION

This application is a continuation-in-part of U.S. application Ser. No. 18/350,663, entitled “MACHINE LEARNING CLOUD BASED CARE PAIRING TECHNOLOGY AND ASSOCIATED METHODS THEREOF,” which was filed on Jul. 11, 2023, and which claims the benefit of and priority to U.S. Patent Application No. 63/368,439, which was filed on Jul. 14, 2022, each of which is incorporated herein by reference in its entirety.

### TECHNICAL FIELD

The present disclosure relates to computing technology for improving mental health services, and more particularly, to providing cloud-based, machine learned models for pairing help seekers with help givers or care providers.

### BACKGROUND

Outside of a help seeker's personal characteristics, the therapeutic alliance, also referred to as a working alliance between a help seeker (also referred to as a patient or a client) and a care provider (also referred to as a help giver, mental health provider or mental health professional) is an important predictor of a positive outcome in psychotherapy (Crits-Christoph, O., Connolly Gibbons, M. B., Hamilton, J., Ring-Kurtz, S., & Gallop, R. (2011). Many help seekers quit therapy after limited sessions, often after only a single session. One reason for this is a perceived “lack of fit” with the mental health provider. Therefore, there is a need for efficient computing technology that can identify patterns to optimize pairing of a help seeker and a mental health provider based upon their collaborative relationship. Continuous efforts are being made to develop such technology.

### BRIEF DESCRIPTION OF THE DRAWINGS

The various features of the present disclosure will now be described with reference to the drawings of the various aspects disclosed herein. In the drawings, the same components may have the same reference numerals. The illustrated aspects are intended to illustrate, but not to limit the present disclosure. The drawings include the following Figures:

FIG. 1 shows an example of an operating environment for the various aspects disclosed herein;

FIG. 2 shows an example of a training process used to develop a predictive machine learned model, according to one aspect of the present disclosure;

FIG. 3 shows an example of a screening process to screen care providers, according to one aspect of the present disclosure;

FIG. 4 shows an example of a pairing process to pair help seekers with care providers, according to one aspect of the present disclosure;

FIG. 5 shows an example of a cloud-based architecture, according to one aspect of the present disclosure;

FIG. 6 shows an example of inputs used to develop the predictive model, according to one aspect of the present disclosure;

FIG. 7 shows an example of a processing system, used according to one aspect of the present disclosure;

FIG. 8 is a simplified block diagram of at least one embodiment of an environment that may be established by a computing system of the system of FIG. 1;

FIG. 9 is a simplified flow diagram of at least one embodiment of a method for clinical outcome predictive analysis with model training that may be executed by the system of FIGS. 1 and 8;

FIG. 10 is a simplified flow diagram of at least one embodiment of a method for clinical outcome predictive analysis with model inference that may be executed by the system of FIGS. 1 and 8;

FIG. 11 is a simplified flow diagram of at least one embodiment of a method for clinical outcome monitoring that may be executed by the system of FIGS. 1 and 8; and

FIG. 12 is a chart illustrating experimental results that may be achieved according to the methods of FIGS. 9-11.

### DETAILED DESCRIPTION

As a preliminary note, the terms “component,” “module,” “system,” and the like as used herein are intended to refer to a computer-related entity, either software-executing general-purpose processor, hardware, firmware and a combination thereof. For example, a component may be, but is not limited to being, a process running on a hardware-based processor, a hardware processor, an object, an executable, a thread of execution, a program, and/or a computer.

By way of illustration, both an application running on a server and the server can be a component. One or more components may reside within a process and/or thread of execution, and a component may be localized on one computer and/or distributed between two or more computers. Also, these components can execute from various non-transitory, computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal).

Computer executable components of the innovative technology disclosed herein can be stored, for example, at a non-transitory, computer readable media including, but not limited to, an ASIC (application specific integrated circuit), CD (compact disc), DVD (digital video disk), ROM (read only memory), storage class memory, solid state drive, floppy disk, hard disk, EEPROM (electrically erasable programmable read only memory), or any other storage device, in accordance with the claimed subject matter.

In one aspect, innovative technology is disclosed herein that uses patterns from machine learning algorithms including demographic, personality trait and/or characteristic variables of a client and a care provider to predict an output of a strong therapeutic/working alliance.

The term “Therapeutic Alliance” (also referred to as a working alliance or alliance) as used herein mean a collaborative relationship, e.g., between a client and a therapist. The alliance defines a collaborative relationship between a help seeker and help giver where there is an agreement on goals, assignment of tasks, and bond development. The alliance is often described between clients and therapists, but it is applicable between other help seeker and help giver relationships including athletes and coaches, patients and care givers, mentees and mentors, students and teachers, and others. Therefore, the terms help giver, mental health provider, care provider includes teachers, coaches, nurses, doctors, mentors, psychologists, and others. The term client includes any help seeker or one who needs mental health services.

The term “Personality Trait” as used herein means a relatively stable, consistent, and enduring internal characteristic that is inferred from a pattern of behaviors, attitudes, feelings, and habits in the individual as defined by the American Psychological Association (APA).

The term “Characteristic(s)” as used herein is a particular feature or quality of a person, especially any of the enduring qualities or traits that define an individual's nature or personality in relation to others as defined by the APA.

In one aspect of the present disclosure, cloud-based computing technology runs machine learning algorithms with inputs of personal attributes of help seekers and help givers to determine a combination of attributes that predict the development of a strong working alliance. The term “helping conversation feedback” as used herein is any feedback from a help seeker that can be used as a proxy for measuring the working alliance, e.g., as defined by Bordin, E. S. (1979) in “The generalizability of the psychoanalytic concept of the working alliance” in Psychotherapy: Theory, Research Practice, 16(3), 252-260. The innovative machine learning model uses help seeker inputs including the Ten-Item-Personality-Inventory (TIPI) (Gosling, S. D., Rentfrow, P. J., & Swann, W. B., Jr. (2003). A very brief measure of the Big Five personality domains. Journal of Research in Personality, 37, 504-528.), and demographic variables together with help giver inputs including the Analogue to Multiple Broadband Inventories (AMBI) personality inventory (Yarkoni, T. (2010). The abbreviation of personality, or how to measure 200 personality scales with 200 items. Journal of Research in Personality, 44, 180-198.), Counselor Activity Self-Efficacy Scales (CASES) (Lent, R. W., Hill, C. E., & Hoffman, M. A. (2003). Development and validation of the Counselor Activity Self-Efficacy Scales. Journal of Counseling Psychology, 50(1), 97-108.), Adult Attachment Questionnaire (AAQ) (Simpson, J. A., Rholes, W. S. & Phillips, D. (1996). Conflicts in close relationships: An attachment perspective. Journal of Personality and Social Psychology, 71(5), 899-914.), and demographic variables to rank help givers based upon predicted strength of the working alliance with a specific help seeker measured by the Session Alliance Inventory (SAI) (Falkenström, F., Hatcher, R. L., Skjulsvik, T., Holmqvist Larsson, M., Holmqvist, R. (2015). Development and validation of a 6-item working alliance questionnaire for repeated administrations during psychotherapy. Psychological Assessment, 27(1), 169-183.). Details of the innovative technology/processes are provided below.

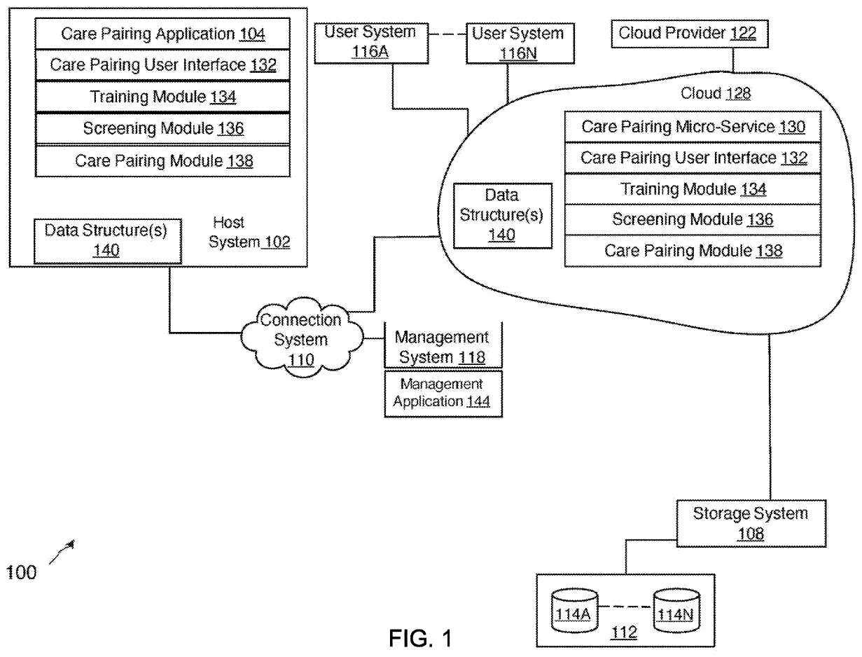

System 100: FIG. 1 shows an example of a system 100, for presenting care pairing micro-services (or microservices, used interchangeably) via a cloud-based system (or “cloud”) 128, according to one aspect of the present disclosure. Cloud 128 provides an abstraction between computing resource and its underlying technical architecture (e.g., servers, storage, networks), enabling convenient, on-demand network access to a shared pool of configurable computing resources that can be rapidly provisioned and released with minimal management effort or service provider interaction. The term “cloud” is intended to refer to a network, for example, the Internet and cloud computing allows shared resources, for example, software and information to be available, on-demand, like a public utility.

Typical cloud computing providers deliver common business applications online which are accessed from another web service or software like a web browser, while the software and data are stored remotely on servers. The cloud computing architecture uses a layered approach for providing application services. A first layer is an application layer that is executed at client computers. In this example, the application allows a client to access storage via a cloud. After the application layer, is a cloud platform and cloud infrastructure, followed by a “server” layer that includes hardware and computer software designed for cloud specific services.

In one aspect, a cloud provider 122 manages access to cloud based services for user systems/devices (or “clients”) 116A-116N that are able to access micro-services, e.g., a care pairing micro-service 130 (may also be referred to as a “micro-service 130” or micro-services 130), described below in detail. User systems 116A-116N include help giver and help seeker systems. Each micro-service 130 includes a User Interface (UI) 132, a training module 134 (also referred to as module 134), a screening module 136 (also referred to as module 136) and a care pairing module 138 (also referred to as module 138). The modules 134, 132 and 18 use one or more application programming interface (“API”) and one or more data structures 140 to execute the innovative care pairing technology of the present disclosure. The term micro-service as used herein denotes computing technology for providing a specific functionality in the networked environment.

System 100 includes a storage system 108 that may be used to store data structures 140 at one or more physical storage sites to store data. A management system 118 executing an application 144 may be used to configure the various components of system 100.

In one aspect, storage system 108 has access to a set of mass storage devices 114A-114N (may also be referred to as storage devices 114) within at least one storage subsystem 112. The mass storage devices 114 may include writable storage device media such as magnetic disks, video tape, optical, DVD, magnetic tape, non-volatile memory devices for example, solid state drives (SSDs) including self-encrypting drives, flash memory devices and any other similar media adapted to store information. The storage devices 114 may be organized as one or more groups of Redundant Array of Independent (or Inexpensive) Disks (RAID). The various aspects disclosed are not limited to any particular storage device type or storage device configuration.

The storage system 108 may be used to store and manage information (e.g. data structures 140) at storage devices 114 based on client requests. The requests may be based on file-based access protocols, for example, the Common Internet File System (CIFS) protocol or Network File System (NFS) protocol, over the Transmission Control Protocol/Internet Protocol (TCP/IP). Alternatively, the request may use block-based access protocols, for example, the Small Computer Systems Interface (SCSI) protocol encapsulated over TCP (iSCSI) and SCSI encapsulated over Fibre Channel (FCP). The adaptive aspects described herein are not limited to any specific request type or request protocol.

In one aspect, system 100 may include one or more computing systems 102 (may also be referred to as host platform(s)/system(s) 102 or simply as server(s) 102) communicably coupled to storage system 108 via a connection system 110 such as a local area network (LAN), wide area network (WAN), the Internet and others. As described herein, the term “communicably coupled” may refer to a direct connection, a network connection, or other connections to enable communication between devices. It is noteworthy that although connection system 110 and cloud 128 are shown as separate entities, functionally, the two systems may be similar in terms of providing access to host systems i.e., the host systems may access the storage systems via cloud 128.

Host system 102 stores instructions in a memory to execute a care pairing application 104 (may also be referred to as application 104) that is like the care pairing micro-service 130 with access to data structures 140. Application 104 and micro-service 130 are referred to interchangeably throughout this specification.

FIG. 1 provides an example of an architecture that enables building complex micro-services application 130 by decomposing them into a set of independent and loosely coupled services. Each service (typically running as one or more operating system processes) provides a specific capability or function and communicates with the other services over a network. Micro-service 130 of the present disclosure is modular, lightweight, and isolated. Microservice 130 can be developed independently and updated or replaced as needed. As an example, micro-service 130 may be deployed as a container (e.g., a “Docker” container described below), is stateless in nature, may be exposed as a API and is discoverable by other services.

The following provides a brief definition of various technologies used for developing and deploying micro-service 130:

“Docker”: Docker is a software framework for building and running micro-services using the Linux kernel (without derogation of any third party trademark rights). The various aspects described herein are not limited to the Linux kernel. In one aspect, Docker micro-service code for micro-service 130 is packaged as a “Docker image file.” A Docker container is then initialized using an associated image file. A Docker container is an active or running instantiation of a Docker image. Each Docker container provides isolation and resembles a lightweight virtual machine. It is noteworthy that many Docker containers can run simultaneously in a same Linux computing system.

Cloud 128 may be based on the Amazon Web Services (AWS) (without derogation of any trademark rights). However, the adaptive aspects may be implemented using other cloud platforms e.g., GOOGLE CLOUD, AZURE and others (without derogation of any third party trademark rights). AWS provides a collection of on-demand public cloud services in different categories, including compute, storage, and database services.

Cloud 128 may also leverage an “AWS Elastic Compute Cloud” that may also be referred to as “AWS EC2,” a service providing scalable compute capacity in the AWS cloud. Micro-service 130 in AWS EC2 is packaged and deployed as an Amazon Machine Image (AMI) file. In one aspect, cloud 128 may include the use of an AWS Virtual Private Cloud (VPC), a service that enables configuration of a logically isolated section of the AWS cloud to simulate a datacenter private cloud.

In one aspect, “Node.js” may be used as a “JavaScript” runtime environment for developing the micro-service 130. AngularJS may be used as a development framework for front-end web applications using Javascript. Code development for micro-service 130 may be controlled using Git repositories. It is noteworthy that these enabling technology examples are simply an illustration. The adaptive aspects of the present disclosure may be implemented using other technologies.

In one aspect, system 100 provides a platform for building and deploying care pairing technology via the cloud. The solution is flexible and extensible, lightweight, rapidly developed, low cost, and adheres to industry standards.

In one aspect, micro-service 130 may exist as an isolated repository, e.g., a local “Git” repository. Git provides technology for developing software code. Git stores data in a data structure called a repository that includes “committed” objects and references to committed objects, called “heads.” When micro-service 130 is ready to be deployed, the local Git repository is pushed to a remote repository for ongoing source code management.

In one aspect, micro-service 130 has its own unique microservices code, which is deployed as several “Docker” containers in a virtual machine. The code for each service can be derived from the samples in the repository. As an example, each service uses a dedicated AWS EC2 virtual machine.

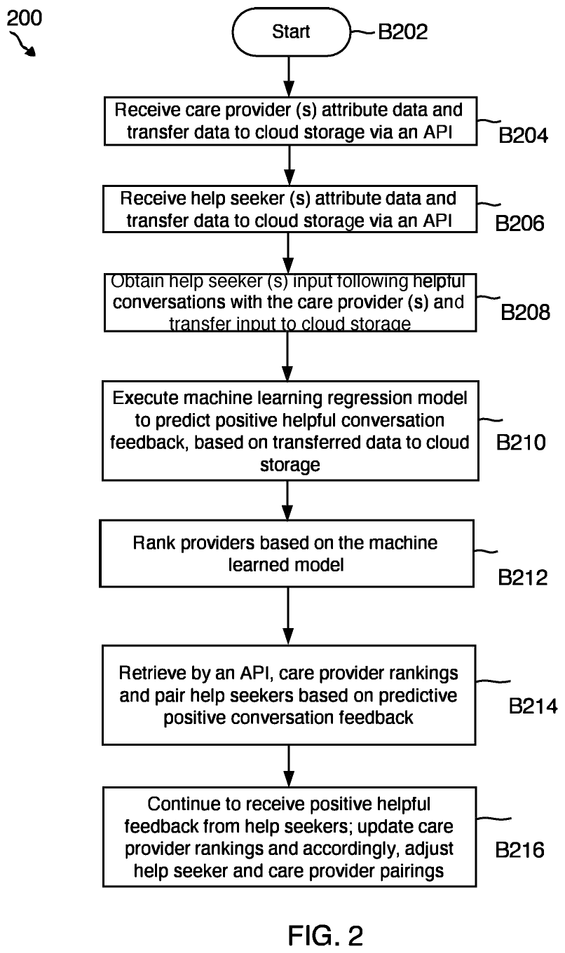

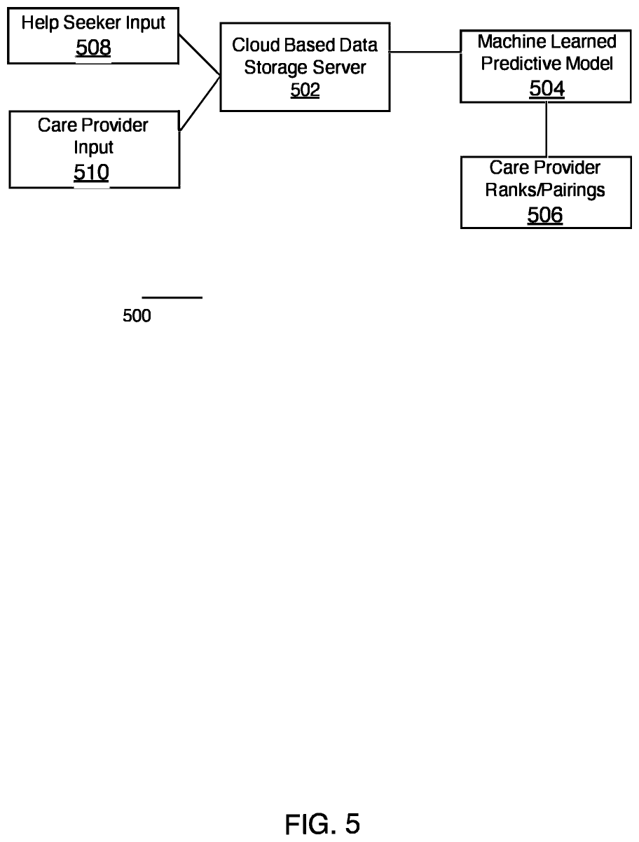

Process Flows: FIG. 2 shows a process 200 for training a machine learned model (e.g., 504, FIG. 5), according to one aspect of the present disclosure. The process begins in block B202, when a computing device (e.g., host 102)/cloud-based system has been initialized to execute the training module 134. In block B204, care provider personality attributes (e.g., 510, FIG. 5) are received and then transferred to cloud-based storage (e.g., to be stored in data structures 140) by an API. The attributes may be received by the host system 102 or directly by the micro-service 130. In block B206, help seeker attributes (e.g., 508, FIG. 5) are received and transferred to cloud-based storage via an API. The attributes may be received by the host system 102 or directly by the micro-service 130.

In block B208, help seeker input is received following helpful conversations with one or more help seekers and then transferred to cloud-based storage (e.g., to be stored in data structures 140) by an API.

In block B210, a machine learning regression model is executed to predict positive helping conversation feedback. As an example, the model is executed by a cloud-based server executing instructions out of a memory (e.g., 502, FIG. 5). The model ranks care providers (e.g., 506, FIG. 5).

In block B214, an API (e.g., the care pairing module 138) retrieves the rankings and pairs help seekers with care providers based on predictive positive conversation feedback. The machine learned model continues to be updated in block B216. The rankings are adjusted as more data is received and pairing may be modified based on the updated model.

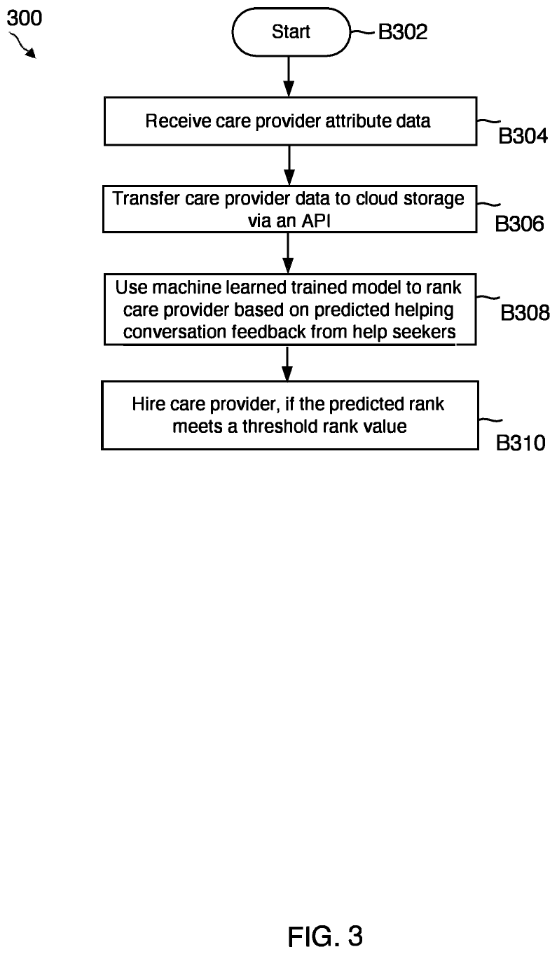

FIG. 3 shows a process 300 executed by module 136, according to one aspect of the present disclosure. The process begins in block B302 when the machine learned model has been developed. In block B304, care provider personality attributes (e.g., 510, FIG. 5) are received and then transferred to cloud based storage (e.g., to data structures 140) by an API in block B306. The attributes may be received by the host system 102 or directly by the micro-service 130.

In block B308, the machine learning regression model 504 is used to rank the care provider based on predicted helping conversation feedback from help seekers. If the rank meets a threshold value, then the care provider is hired in block B310.

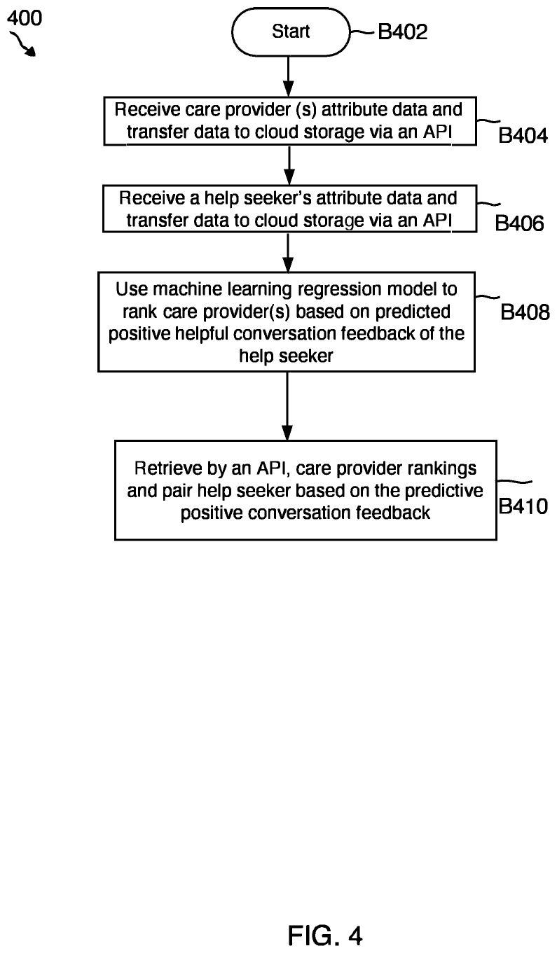

FIG. 4 shows a process 400 executed by module 138 for pairing a help seeker with care provider using a machine learned model (e.g., 504, FIG. 5), according to one aspect of the present disclosure. The process begins in block B402, when a computing device/cloud-based system has been initialized to execute the training module 134.

In block B404, care provider personality attributes (e.g., 510, FIG. 5) are received and then transferred to cloud-based storage (e.g., data structures 140) by an API. The attributes may be received by the host system 102 or directly by the micro-service 130.

In block B406, a help seeker's attributes (e.g., 508, FIG. 5) are received and transferred to cloud-based storage via an API. The attributes may be received by the host system 102 or directly by the micro-service 130.

In block B408, the machine learning regression model is used to rank care providers based on predicted positive helping conversation feedback of the help seeker.

In block B410, an API (e.g., module 138) retrieves the rankings and pairs the help seeker with a care provider based on predictive positive conversation feedback. The machine learned model continues to be updated. The rankings are adjusted as more data is received and the pairing may be modified based on the updated model.

Cloud Environment: FIG. 5 shows a cloud environment 500 to implement the various aspects of the present disclosure. The cloud environment 500 includes a cloud-based server 502 that has access to help seeker input 508 and care provider input 510. As described above with respect to FIGS. 2-4, the cloud-based server 502 executes the machine learned model 504 to generate care provider rankings 506 to pair help seekers with care providers based on the rankings.

As an example, inputs 508 and 510 may be collected through a Vue.js front end and a Node.js back end with data encoded and stored as JSON (JavaScript Object Notation) files. Amazon DynamoDB may be used as the database with ML model 504 written in Python and running through Amazon Sagemaker (without derogation of any Amazon trademark rights).

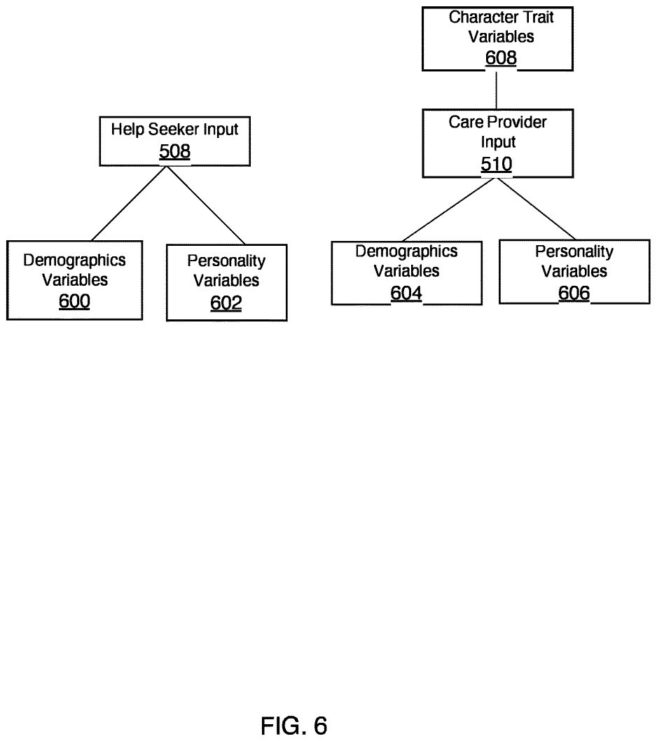

FIG. 6 shows an example of help seeker input 508 and care provider input 510. The help seeker input includes demographics data 600 and personality variables 602, while the care provider input includes demographics 604, personality variables 606 and character trait variables 608. These inputs can be stored in data structures 140 and used by the process blocks of FIGS. 2-4.

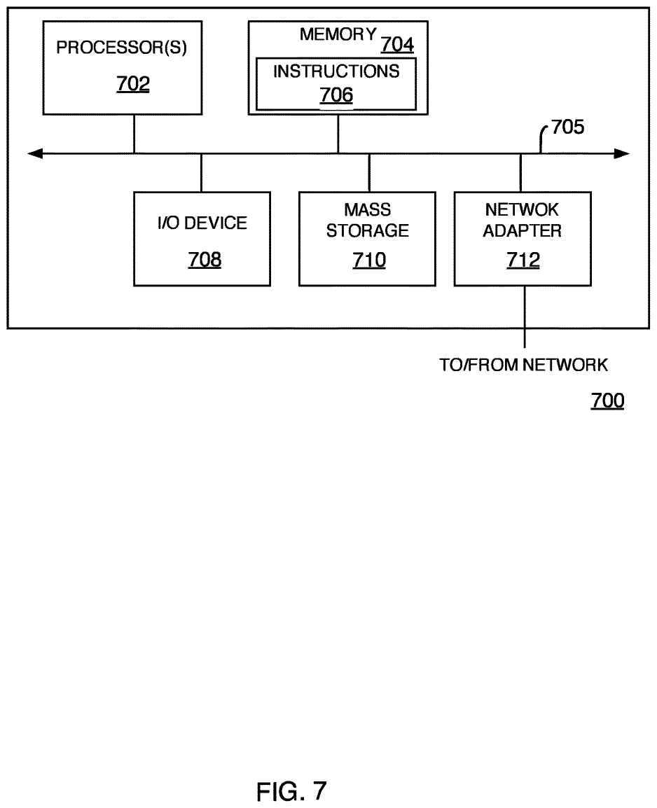

Processing System: FIG. 7 is a high-level block diagram showing an example of the architecture of a processing system 700 that may be used according to one aspect. The processing system 700 can represent host system 102, management system 118, user systems 116, cloud provider 122, storage system 108, and a cloud-based device (e.g., 502, FIG. 5) hosting the micro-services 130. Note that certain standard and well-known components which are not germane to the present aspects are not shown in FIG. 7.

The processing system 700 includes one or more processor(s) 702 and memory 704, coupled to a bus system 705. The bus system 705 shown in FIG. 7 is an abstraction that represents any one or more separate physical buses and/or point-to-point connections, connected by appropriate bridges, adapters and/or controllers. The bus system 705, therefore, may include, for example, a system bus, a Peripheral Component Interconnect (PCI) bus, a HyperTransport or industry standard architecture (ISA) bus, a small computer system interface (SCSI) bus, a universal serial bus (USB), or an Institute of Electrical and Electronics Engineers (IEEE) standard 1394 bus (sometimes referred to as “Firewire”).

The processor(s) 702 are the central processing units (CPUs) of the processing system 700 and, thus, control its overall operation. In certain aspects, the processors 702 accomplish this by executing software stored in memory 704. A processor 702 may be, or may include, one or more programmable general-purpose or special-purpose microprocessors, digital signal processors (DSPs), programmable controllers, application specific integrated circuits (ASICs), programmable logic devices (PLDs), or the like, or a combination of such devices.

Memory 704 represents any form of random access memory (RAM), read-only memory (ROM), flash memory, or the like, or a combination of such devices. Memory 704 includes the main memory of the processing system 700. Instructions 706 implement the process steps of FIGS. 2-4 and data structures 140 described above may reside in and execute (by processors 702) from memory 704.

Also connected to the processors 702 through the bus system 705 are one or more internal mass storage devices 710, and a network adapter 712. Internal mass storage devices 710 may be or may include any conventional medium for storing data in a non-volatile manner. The network adapter 712 provides the processing system 700 with the ability to communicate with remote devices (e.g., storage servers) over a network and may be, for example, an Ethernet adapter, a Fibre Channel adapter, or the like.

The processing system 700 also includes one or more input/output (I/O) devices 708 coupled to the bus system 705. The I/O devices 708 may include, for example, a display device, a keyboard, a mouse, etc.

Thus, innovative care paring technology in a cloud-based system has been described. Note that references throughout this specification to “one aspect” (or “embodiment”) or “an aspect” mean that a particular feature, structure or characteristic described in connection with the aspect is included in at least one aspect of the present disclosure. Therefore, it is emphasized and should be appreciated that two or more references to “an aspect” or “one aspect” or “an alternative aspect” in various portions of this specification are not necessarily all referring to the same aspect. Furthermore, the particular features, structures or characteristics being referred to may be combined as suitable in one or more aspects of the disclosure, as will be recognized by those of ordinary skill in the art.

While the present disclosure is described above with respect to what is currently considered its preferred aspects, it is to be understood that the disclosure is not limited to that described above. To the contrary, the disclosure is intended to cover various modifications and equivalent arrangements within the spirit and scope of the appended claims.

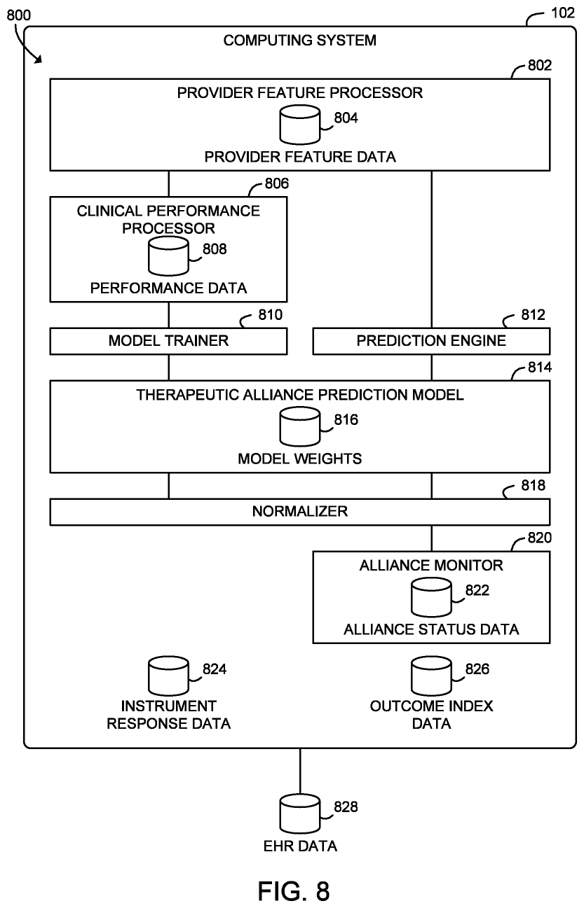

Referring now to FIG. 8, in another illustrative embodiment, the computing system 102 establishes an environment 800 during operation. The illustrative environment 800 includes a provider feature processor 802, a clinical performance processor 806, a model trainer 810, a prediction engine 812, a therapeutic alliance prediction model 814, a normalizer 818, and an alliance monitor 820. The various components of the environment 800 may be embodied as hardware, firmware, software, or a combination thereof. As such, in some embodiments, one or more of the components of the environment 800 may be embodied as circuitry or a collection of electrical devices (e.g., provider feature processor circuitry 802, clinical performance processor circuitry 806, model trainer circuitry 810, prediction engine circuitry 812, therapeutic alliance prediction model circuitry 814, normalizer circuitry 818, and/or alliance monitor circuitry 820). It should be appreciated that, in such embodiments, one or more of those components may form a portion of the processor 702, the memory 704, the bus system 705, the mass storage device 710, and/or other components of the computing system 102.

The provider feature processor 802 is configured to extract multiple provider features 804 from an assessment instrument. The provider features 804 may be extracted, for example, from provider responses or other data stored in instrument response data 824. The provider features 804 are indicative of relational characteristics of a care provider (e.g., a therapist, mental health provider, mental health professional, or other help giver). In some embodiments, the assessment instrument integrates multiple validated psychometric instruments. In some embodiments, the relational characteristics are indicative of attachment style, self-efficacy, and personality characteristics of the care provider. In some embodiments, the provider features 804 include multiple composite or aggregate scores that are each associated with a domain.

The clinical performance processor 806 is configured to determine a clinical outcome performance metric 808 associated with the care provider. In some embodiments, the clinical outcome performance metric may be determined by retrieving the clinical outcome performance metric from an organization electronic health records (EHR) system, or otherwise connecting with EHR data 828. In some embodiments, the clinical outcome performance metric is indicative of a predetermined key performance indicator (KPI), such as, for example, patient engagement, treatment completion, and discharge against medical advice (AMA). In some embodiments, the clinical outcome performance metric is a composite z-score based on multiple composite or aggregate scores of the provider features 804.

The model trainer 810 is configured to link the provider features 804 and the clinical outcome performance metric 808 in training data and to train the therapeutic alliance prediction model 814 with the training data to predict an outcome score based on provider feature. The outcome score is indicative of the clinical outcome performance metric. The therapeutic alliance prediction model 814 is a machine learning model, and in the illustrative embodiment is a machine learning regression model. For example, the prediction model 814 may be embodied as an XGBoost or other gradient boosted decision tree model, a random forest model, and/or other appropriate prediction model. Accordingly, the model 814 includes multiple weights 816, which are adjusted during model training.

The normalizer 818 is configured to determine a normalization factor for the predicted outcome performance metric to a predicted outcome index. The predicted outcome index is illustratively a single numerical value within a predetermined range, such as 0 to 100. The normalizer 818 is further configured to normalize a predicted outcome score to a predicted outcome index as a function of the normalization factor. The normalized, predicted outcome index may be stored in outcome index data 826.

The prediction engine 812 is configured to predict a predicted outcome score based on provider features 804 using the prediction model 814 after training of the prediction model 814. As described above, the predicted outcome score is indicative of a clinical outcome performance metric, and the prediction model 814 is trained to predict a predicted outcome score based on provider features 804.

The alliance monitor 820 is configured to pair a client to a particular care provider based on the predicted outcome index associated with that particular care provider. The alliance monitor 820 is further configured to monitor for ongoing therapeutic alliance status data 822 associated with the paired client and care provider, and to predict a risk of alliance failure based on the ongoing therapeutic alliance status data 822 using a pretrained prediction model. In some embodiments, monitoring for the ongoing therapeutic alliance status data includes connecting to an organization electronic health records system, such as the EHR data 828. In some embodiments, the ongoing therapeutic alliance status data 822 includes attendance patterns, engagement with treatment plans, symptom changes, and/or client feedback. In some embodiments, the alliance monitor 820 is configured to automatically assess whether predicted outcome scores from the trained prediction model 814 maintain a consistent relationship with performance indicators determined based on the ongoing therapeutic alliance status data 822. In some embodiments, automatically assessing the trained prediction model 814 includes applying a predetermined statistical method to the ongoing therapeutic alliance status data 822.

Although FIG. 8 illustrates the environment 800 as including all of the provider feature processor 802, the clinical performance processor 806, the model trainer 810, the prediction engine 812, the therapeutic alliance prediction model 814, the normalizer 818, and the alliance monitor 820, it should be understood that in some embodiments one or more of those components may be established by different computing systems 102, and the operations of those components may be distributed in space and/or in time. For example, in an embodiment the provider feature processor 802, the clinical performance processor 806, and the model trainer 810 may be established by a computing system 102 configured to perform model training, for example in a cloud computing environment. Continuing that example, the provider feature processor 802, the prediction engine 812, and the normalizer 818 may be established by a computing system 102 configured to perform model inference, for example on a workstation or in a cloud environment. The therapeutic alliance prediction model 814 and/or the model weights 816 may be provisioned to the computing system 102 configured to perform model inference. Continuing that example further, the therapeutic alliance prediction model 814 may be established by a separate computing system 102 to perform inference, for example in a cloud computing environment. As yet another example, the alliance monitor 820 may be established by the same computing system 102 configured to perform model inference and/or a different computing system 102 configured to monitor alliance status data. Of course, other configurations of computing devices are possible in other environments.

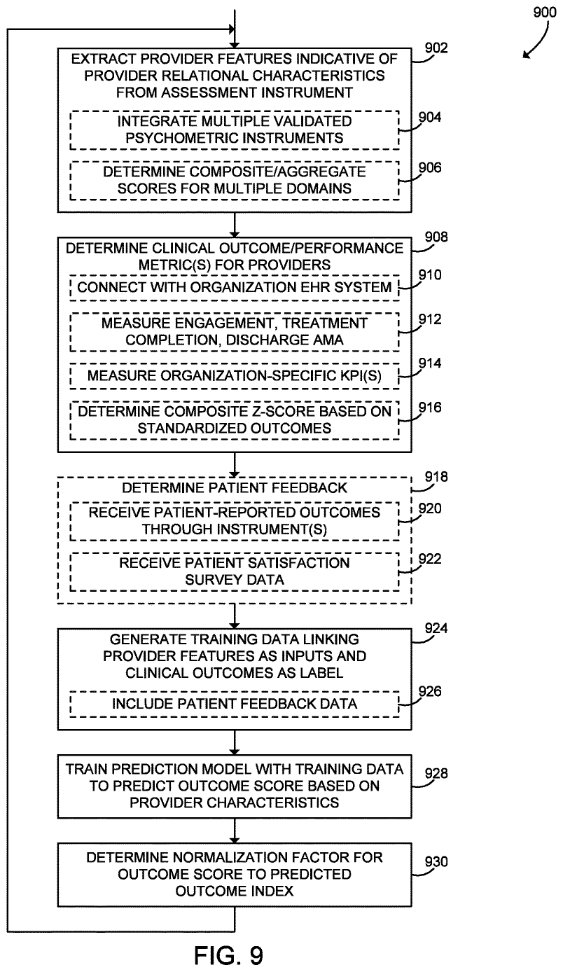

Referring now to FIG. 9, in use, the computing system 102 may execute a method 900 for clinical outcome predictive analysis with model training. It should be appreciated that, in some embodiments, the operations of the method 900 may be performed by one or more components of the environment 800 of the computing system 802 as shown in FIG. 8. The method 900 begins with block 902, in which the computing system 102 extracts provider features 804 indicative of provider relational characteristics from assessment instrument responses 824. The instrument responses 824 may include response data from multiple providers (e.g., therapists or other help givers) associated with an organization (e.g., a clinic or other organization) who have completed a set of one or more assessment instruments. The provider features 804 include indicators of attachment security, relational consistency, and clinical performance. The computing system 102 may transform raw responses 824 to the instrument into structured behavioral trait features 804. Accordingly, those features 804 represent stable behavioral and cognitive tendencies of the provider, rather than demographic or experiential background of the provider. In the illustrative embodiment, the provider features 804 are related to a particular provider at a particular point in time (e.g., based on one set of responses 824 to the instrument). Additionally or alternatively, in some embodiments, extraction of provider features 804 may be repeated over time, for example in longitudinal designs. In some embodiments, in block 904 the computing system 102 integrates multiple validated psychometric instruments. For example, as described further below, the computing system 102 may integrate three such validated psychometric instruments. Accordingly, the computing system 102 integrates multiple measures of attachment style, self-efficacy, and personality characteristics. Provider attachment style may be a predictor for therapeutic alliance strength. In some embodiments, in block 906 the computing system 102 determines composite and/or aggregate scores for multiple domains. For example, the computing device 102 may determine subdomain composite scores (e.g., Empathy, Conscientiousness, Cognitive Flexibility, Stress Tolerance) and/or domain-level aggregates (e.g., Interpersonal Effectiveness, Task Orientation, Emotional Regulation). For example, in the illustrative embodiment the computing system 102 scores responses to a 234-item instrument across 24 domains. The score for each domain may incorporate percentile ranks and/or otherwise scaled scores. In some embodiments, the computing device 102 may also determine response-style indicates (e.g., extreme responding, acquiescence controls where applicable). Accordingly, the computing system 102 provides a multi-domain psychometric assessment.

As an illustrative example, and as described above, in an embodiment a primary assessment tool may include a 234-item instrument designed to measure 24 provider characteristics that have been empirically linked to therapeutic alliance and patient outcomes. An abbreviated 192-item version of this instrument is also available for non-counseling roles. Together, these versions generate comprehensive profiles of care providers, offering insight into their interpersonal, cognitive, and relational tendencies.

The illustrative instrument integrates items from three established psychometric instruments to provide a holistic view of providers' relational and professional capacities. First, the Adult Attachment Questionnaire (AAQ) evaluates attachment style, a central determinant of relational security, empathy, and flexibility in therapeutic interactions. The illustrative questionnaire includes items such as:
I find it relatively easy to get close to others.
I find it difficult to trust others completely.

Second, the Counselor Activity Self-Efficacy Scales (CASES) scale assesses providers' self-efficacy across a broad range of counseling activities, reflecting their confidence and perceived competence in clinical practice. This instrument assesses provider skills, such as listening and asking open questions; evaluates provider effectiveness on clinical tasks, such as keeping sessions focused and building a clear conceptualization of client issues; and measures provider self-confidence in working effectively with different client types (for example, suicidal, sexually abused), client issues (such as those the provider might find personally difficult), and scenarios (such as where the provider or client might find the other sexually attractive). Third, the Analog to Multi-Broadband Inventories (AMBI) instrument is a public domain, 181-item personality inventory designed to simultaneously capture the relational traits measured by eight different established broadband personality tests using a significantly shorter assessment length. It captures relational traits across hundreds of scales, identifying empirically derived predictors of alliance strength and treatment outcomes.

The combined use of these measures enables the illustrative instrument to generate a multidimensional profile of a provider's personality, self-efficacy, and attachment-related behaviors-which are factors shown to directly influence treatment engagement and retention.

In block 908, the computing system 102 determines clinical outcome and/or performance metric(s) 808 for providers. The performance metric(s) 808 define what success means in a particular organization's operational environment. Each of the clinical outcome/performance metrics 808 may be stored per-provider, and thus can be anonymized or otherwise aggregated across patients. In some embodiments, in block 910 the computing system 102 connects with an organization electronic health records (EHR) system. The EHR system may include an external server, an on-premises server, and/or other EHR data source 828. In some embodiments, in block 912 the computing system 102 measures patient engagement, treatment completion, and/or discharge against medical advice (AMA) as part of the clinical outcome and/or performance metrics 808. In some embodiments, in block 914 the computing system 102 measures one or more organization-specific key performance indicators (KPIs) as part of the outcome and/or performance metrics 808. These KPIs may be specified by the organization associated with the EHR data 828. For example, typical categories for performance metrics 808 may include operational productivity metrics (e.g., caseload volume, patients served, visit frequency, and/or revenue or billing productivity), clinical/quality outcomes when available (e.g., PHQ-9 improvement, GAD-7 improvement, treatment completion, and/or retention duration), administrative/process metrics (e.g., days authorized, days retrobilled, documentation compliance, and/or authorization approval rates), employment outcomes (e.g., tenure/retention length and/or promotion or performance tier assignment), and/or other performance metrics. Accordingly, the clinical outcome/performance metric 808 may include an organization-specific composite success metric. In some embodiments, in block 916 the computing system 102 determines a composite z-score based on standardized outcomes. This composite z-score may be used as a single-value clinical outcome/performance metric 808 per provider. The composite score may be standardized, direction-corrected, and combined as a weighted score. In an illustrative embodiment, each KPI is given equal weight. Additionally or alternatively, in some embodiments, the weights for each KPI may be organization-prioritized, empirically adjusted, or otherwise determined.

In some embodiments, in block 918 the computing system 102 determines patient feedback. In some embodiments, in block 920 the computing system 102 receives patient-reported outcome data through one or more instrument(s). In some embodiments, in block 922 the computing system 102 receives patient satisfaction survey data. For example, to validate and refine the predictive capacity of the prediction model 814, the training data is linked with patient-reported outcomes collected through clinically validated instruments, including: Generalized Anxiety Disorder scale (GAD-7), Patient Health Questionnaire (PHQ-9), Brief Revised Working Alliance Inventory (BR-WAI), Personal Wellbeing Index (PWI), and/or Life Satisfaction Survey. Additionally, the computing system 102 may incorporate feedback from client satisfaction surveys, ensuring the representation of both clinical and experiential perspectives in the evaluation of provider effectiveness. These data sources allow for robust outcome modeling, linking provider characteristics to both symptom reduction and patient-perceived quality of care. Such ongoing data modeling using patient outcome data and feedback may identify additional traits influencing therapeutic success, thereby refining predictive accuracy over time.

In block 924, the computing system 102 generates labeled training data using the provider features 804 as inputs and the clinical outcome/performance data 808 as labels. The provider features 804 and the clinical outcome/performance data 808 may be joined at the provider level. That is, outcome/performance data 808 associated with a particular provider are used to label provider features 804 associated with that provider. For example, in an illustrative embodiment, each row in a modeling dataset or other training data represents input features 804 and target metrics 808 indexed for one unique individual. In some embodiments, in block 926 the computing system 102 may include patient feedback data as labels in the training data.

In an illustrative embodiment, assembly of training data may include one or more deterministic and/or probabilistic matching operations. For example, the computing system 102 may assemble training data by performing case normalization (upper/lowercase standardization), whitespace and punctuation stripping, standardized full-name field creation, Separate first/last name fields for alternative matching, and/or optional fuzzy matching for edge cases (with manual QA only when necessary). In an embodiment, only high-confidence matches are retained.

In an embodiment, organization data (e.g., data retrieved from EHR data 828 or other organization data) may be cleaned or otherwise preprocessed using one or more operations including missingness analysis per KPI, outlier detection (e.g., extreme tenure or volume anomalies), unit harmonization (e.g., converting percentages vs. counts), and/or consistency checks across time windows. A unified training set is built linking the assessment features 804 and the outcome metrics 808 at the individual level.

Because KPIs exist on different scales, in an embodiment, continuous KPIs are z-scored within the organization sample. Directionality is aligned (e.g., higher value corresponds to better performance). When needed, bounded transformations (e.g., log scaling) may be performed to reduce skew. This allows for a fair combination of KPIs into composite success metrics 808. In an embodiment, multiple z-scores z₁ to zn may be combined into a composite score using corresponding weights w₁ to wn.

$${z}_{composite} = {w}_{1} {z}_{1} + {w}_{2} {z}_{2} + … + {w}_{n} {z}_{n}$$

In some embodiments, KPIs may be excluded if they are statistically independent of behavioral constructs, structurally biased by external factors (e.g., insurance mix), highly sparse or unstable, and/or conceptually contradictory. Refinements may be documented and aligned with organizational goals.

In block 928, the computing system 102 trains the therapeutic alliance prediction model 814 with the training data to predict an outcome score based on provider characteristics. As described above, the prediction model 814 may be embodied as a machine learning regression model. Accordingly, the computing system 102 may use any appropriate machine learning training algorithm to train the prediction model 814. For example, the computing device 102 may perform training using randomized train/test splits with K-fold cross-validation and bootstrapping for coefficient/feature stability. After training, the predicted outcome score may be, for example, a vector of provider domain scores, a predicted composite z-score, and/or other predicted numerical score based on the outcome/performance data 808 as described above. As described above, the patient features 804 extracted from the instrument response data 824 are indicative of provider characteristics.

In block 930, the computing device 102 determines a normalization factor used to normalize the predicted outcome score to a predicted outcome index. The predicted outcome index may be normalized to any predetermined range or scale of numeric scores, which may ease interpretability of the outcome index. In the illustrative embodiment, the predicted outcome index is normalized to a predetermined scale between 0 and 100, centered around 70. In the illustrative embodiment, the predicted outcome score is normalized using theoretical minimum and maximum scores, which may ensure cross-cohort comparability, stability over time, and/or resistance to cohort-specific compression. After determining the normalization factor, the method 900 loops back to block 902 to continue extracting provider features 804 and training the prediction model 814. For example, the computing system 102 may perform periodic performance snapshot training and/or or continuous live enrichment of the prediction model 814.

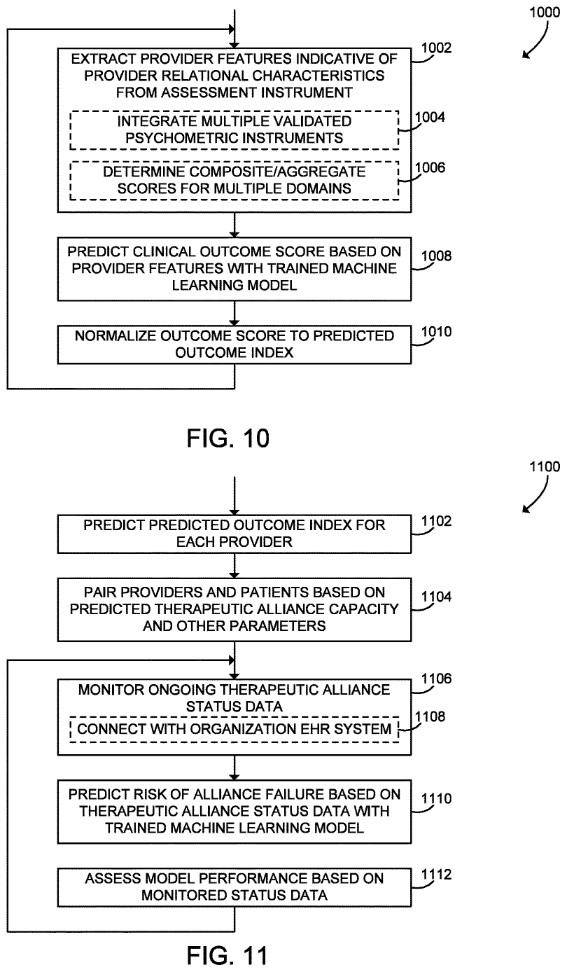

Referring now to FIG. 10, in use, the computing system 102 may execute a method 900 for clinical outcome predictive analysis with model inference. It should be appreciated that, in some embodiments, the operations of the method 1000 may be performed by one or more components of the environment 800 of the computing system 802 as shown in FIG. 8. The method 1000 begins with block 1002, in which the computing system 102 extracts provider features 804 indicative of provider relational characteristics from assessment instrument responses 824 for a particular provider. As described above, the provider features 804 include indicators of attachment security, relational consistency, and clinical performance. In some embodiments, in block 1004 the computing system 102 integrates multiple validated psychometric instruments, as described above. Accordingly, the computing system 102 integrates multiple measures of attachment style, self-efficacy, and personality characteristics. In some embodiments, in block 1006 the computing system 102 determines composite and/or aggregate scores for multiple domains. For example, in the illustrative embodiment the computing system 102 scores responses to a 234-item instrument across 24 domains. The score for each domain may incorporate percentile ranks and/or otherwise scaled scores. Accordingly, the computing system 102 provides a multi-domain psychometric assessment.

In block 1008, the computing system 102 predicts a clinical outcome score based on the provider features 804 with the trained therapeutic alliance prediction model 814. As described above, the therapeutic alliance prediction model 814 is trained to predict an outcome score based on provider characteristics represented in the provider features 804. The predicted outcome score generated by the prediction model 814 may be, for example, a vector of provider domain scores, a predicted composite z-score, and/or other predicted numerical score similar to the outcome/performance data 808 as described above.

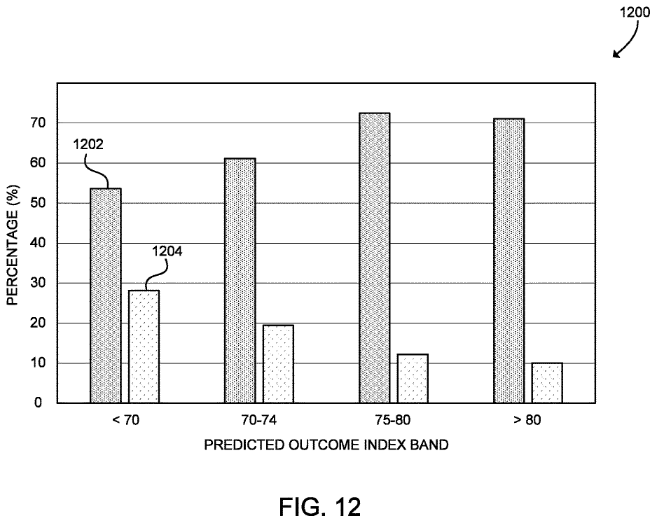

In block 1010, the computing system 102 normalizes the predicted outcome score to generate a predicted outcome index, which may be stored in outcome index data 826. The predicted outcome score may be normalized, for example, as a function of a predetermined normalization factor determined as described above in connection with FIG. 9. As described above, the predicted outcome index may be normalized to any predetermined range or scale of numeric scores, which may ease interpretability of the outcome index. In the illustrative embodiment, the predicted outcome index is normalized to a predetermined scale between 0 and 100, centered around 70. As described above, in the illustrative embodiment, the predicted outcome score is normalized using theoretical minimum and maximum scores. As shown by illustrative experimental results in FIG. 12 and as described further below, a higher predicted outcome index for a particular provider is associated with higher clinical outcome metrics for that provider. For example, it has been experimentally demonstrated that patients whose provider had a predicted outcome index of 70 or higher demonstrated substantially better treatment outcomes compared with those whose providers scored below 70. After generating the normalized, predicted outcome index, the method 1000 loops back to block 1002, in which the computing device 102 may continue to extract provider features 804 and perform inference to generate predicted outcome index.

Referring now to FIG. 11, in use, the computing system 102 may execute a method 1100 for clinical outcome monitoring. It should be appreciated that, in some embodiments, the operations of the method 1100 may be performed by one or more components of the environment 800 of the computing system 802 as shown in FIG. 8. The method 1100 begins with block 1102, in which the computing system 102 predicts a predicted outcome index for each provider at an organization. The computing system 102 may, for example, predict the outcome index using the trained prediction model 814 as described above in connection with the method of FIG. 10.

In block 1104, the computing system 102 pairs providers and patients based on predicted therapeutic alliance capacity, as represented by the predicted outcome index, and one or more other parameters. In block 1106, the computing system 102 monitors ongoing therapeutic alliance status data 822 associated with pairings of patients with providers. The monitored therapeutic alliance status data 822 may include, for example, attendance patterns, engagement with treatment plans, symptom changes, client feedback, and/or other data related to ongoing treatment. In some embodiments, in block 1108 the computing system 102 connects with an organization electronic health records (EHR) system to monitor the ongoing therapeutic alliance status data 822. As described above, the EHR system may include an external server, an on-premises server, and/or other EHR data source 828.

In block 1110, the computing system 102 predicts risk of alliance failure based on the therapeutic alliance status data 822 using a trained machine learning model. The model may be trained, for example, on historical clinical outcome data for similar patients. In an illustrative example, increased risk of alliance failure was predicted multiple weeks before clients dropped out of treatment (e.g., 2-4 weeks in one experiment). In response to an increased risk of alliance failure, a provider may have opportunity to step in, address problems, and keep patients engaged. In an embodiment, the computing system 102 may suggest one or more specific, evidence based strategies tailored to identified risk factors, drawing from validated rupture resolution protocols.

In block 1112, the computing system 102 assesses performance of the therapeutic alliance prediction model 814 using monitored clinical outcome data. For example, in embodiment the computing system 102 performs automated validation of the prediction model 814 and/or other predictive scoring models using analysis scripts, structured data inputs, and execution logic that periodically evaluates the correlation between predicted outcome index scores and defined outcome metrics. The illustrative computing system 102 functions as a repeatable testing environment that applies standardized statistical methods to assess whether predicted outcome index scores maintain a consistent relationship with performance indicators, such as treatment completion rates, against medical advice (AMA) discharge rates, and other outcome measures. After predicting risk of alliance failure and assessing model performance, the method 1100 loops back to block 1106 to continue monitoring the ongoing therapeutic alliance status data 822.

Referring now to FIG. 12, diagram 1200 illustrates experimental results that may be achieved according to the methods of FIGS. 9-11. In an experiment, data was collected from five addiction and mental health treatment organizations across the United States. The study sites represented a range of program types, geographic regions, and treatment philosophies. The diversity across these sites allowed the study to capture provider profiles and patient outcomes across varied organizational models, treatment philosophies, and client populations. The dataset included 6-12 months of treatment records for patients served by therapists across the five organizations. Predicted outcome index scores were linked to patient-level outcomes (illustratively, treatment completion and against medical advice (AMA) discharges). A predicted outcome index score of 70 or higher was considered “ideal.”

The diagram 1200 illustrates experimental results across bands of predicted outcome index score. Series 1202 represents completion rate in percentage, with a higher completion rate corresponding to higher performance. Series 1204 represents AMA discharge rate in percentage, with a lower AMA discharge rate corresponding to higher performance. The series 1202, 1204 are illustratively grouped into for bands: those with providers having a predicted outcome index score of less than 70, those with providers having a predicted outcome index score between 70-74, those with providers having a predicted outcome index score between 75-80, and those with providers having a predicted outcome index score greater than 80. As shown, the highest completion rates were observed among patients treated by providers with predicted output index scores between 75-80(72.47%), whereas AMA discharge rates were lowest among therapists scoring above 80(10.07%). This pattern suggests that incremental improvements in predicted outcome index scores may yield measurable benefits for patient outcomes.

Thus, experimental findings demonstrate that therapists' interpersonal and clinical profiles, as measured by the predicted outcome index, are significantly associated with improved patient outcomes. Higher predicted outcome index scores were associated with both increased treatment completion and reduced AMA discharges. These findings align with prior evidence that therapist characteristics and attachment styles are central to building and sustaining therapeutic alliance. In substance use disorder (SUD) treatment, where dropout rates remain a critical barrier to recovery, these findings highlight the tangible impact of provider relational qualities on patient retention.

The assessment tool used in this study, the predicted outcome index (also called the Care Predictor Index (CPI), represents a novel approach to measuring provider characteristics that influence therapeutic alliance. It is a scientifically validated tool that integrates indicators of attachment security, relational consistency, and clinical performance. The tool has been validated to reduce rates of treatment dropout or discharge against medical advice while improving treatment completion rates and patient engagement.

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

### EXAMPLES

Example 1 includes a computing device for clinical outcome predictive analysis, the computing device comprising: a provider feature processor configured to extract a plurality of provider features from an assessment instrument, wherein the provider features are indicative of relational characteristics of a care provider; a clinical performance processor configured to determine a clinical outcome performance metric associated with the care provider; a model trainer configured to (i) link the plurality of provider features and the clinical outcome performance metric in training data and (ii) train a prediction model with the training data to predict an outcome score based on provider feature, wherein the outcome score is indicative of the clinical outcome performance metric; and a normalizer configured to determine a normalization factor for the predicted outcome performance metric to a predicted outcome index.

Example 2 includes the subject matter of Example 1, and wherein the assessment instrument integrates a plurality of validated psychometric instruments.

Example 3 includes the subject matter of any of Examples 1 and 2, and wherein the relational characteristics are indicative of attachment style, self-efficacy, and personality characteristics of the care provider.

Example 4 includes the subject matter of any of Examples 1-3, and wherein the plurality of provider features comprises a plurality of composite or aggregate scores that are each associated with a domain.

Example 5 includes the subject matter of Example 4, and wherein the clinical outcome performance metric comprises a composite z-score based on the plurality of composite or aggregate scores.

Example 6 includes the subject matter of any of Examples 1-5, and wherein to determine the clinical outcome performance metric comprises to retrieve the clinical outcome performance metric from an organization electronic health records system.

Example 7 includes the subject matter of Example 6, and wherein the clinical outcome performance metric is indicative of a predetermined key performance indicator.

Example 8 includes the subject matter of any of Examples 1-7, and wherein the clinical outcome performance metric is indicative of patient engagement, treatment completion, and discharge against medical advice.

Example 9 includes the subject matter of any of Examples 1-8, and wherein the prediction model comprises a machine learning regression model.

Example 10 includes the subject matter of any of Examples 1-9, and further comprising a prediction engine, wherein: the provider feature processor is further configured to extract a second plurality of provider features from the assessment instrument, wherein the second provider features are indicative of relational characteristics of a second care provider; the prediction engine is configured to predict a predicted outcome score based on the second plurality of provider features using the prediction model in response to training of the prediction mode; and the normalizer is further configured to normalize the predicted outcome score to a first predicted outcome index as a function of the normalization factor.

Example 11 includes computing device for clinical outcome predictive analysis, the computing device comprising: a provider feature processor configured to extract a plurality of provider features from an assessment instrument, wherein the provider features are indicative of relational characteristics of a first care provider; a prediction engine configured to predict a predicted outcome score based on the plurality of provider features with a pre-trained prediction model, wherein the predicted outcome score is indicative of a clinical outcome performance metric, and wherein the prediction model is trained to predict a predicted outcome score based on provider features; and a normalizer configured to normalize the predicted outcome score to a first predicted outcome index.

Example 12 includes the subject matter of Example 11, and wherein the assessment instrument integrates a plurality of validated psychometric instruments.

Example 13 includes the subject matter of any of Examples 11 and 12, and wherein the relational characteristics are indicative of attachment style, self-efficacy, and personality characteristics of the care provider.

Example 14 includes the subject matter of any of Examples 11-13, and wherein the plurality of provider features comprises a plurality of composite or aggregate scores that are each associated with a domain.

Example 15 includes the subject matter of Example 14, and wherein the clinical outcome performance metric comprises a composite z-score based on the plurality of composite or aggregate scores.

Example 16 includes the subject matter of any of Examples 11-15, and wherein to normalize the predicted outcome score comprises to normalize the predicted outcome score as a function of a predetermined normalization factor.

Example 17 includes the subject matter of any of Examples 11-16, and wherein the pre-trained prediction model comprises a machine learning regression model.

Example 18 includes the subject matter of any of Examples 11-17, and further comprising an alliance monitor configured to pair a first client to the first care provider based on the first predicted outcome index.

Example 19 includes the subject matter of Example 18, and wherein the alliance monitor is further configured to: monitor for ongoing therapeutic alliance status data associated with the first client and the first care provider; and predict a risk of alliance failure based on the ongoing therapeutic alliance status data using a second pretrained prediction model.

Example 20 includes the subject matter of Example 19, and wherein to monitor for the ongoing therapeutic alliance status data comprises to connect to an organization electronic health records system.

Example 21 includes the subject matter of any of Examples 19 and 20, and wherein the ongoing therapeutic alliance status data comprises attendance patterns, engagement with treatment plans, symptom changes, or client feedback.

Example 22 includes the subject matter of any of Examples 19-21, and wherein the alliance monitor is further configured to automatically assess whether predicted outcome scores from the trained prediction model maintain a consistent relationship with performance indicators determined based on the ongoing therapeutic alliance status data.

Example 23 includes the subject matter of Example 22, and wherein to automatically assess the trained prediction model compress to apply a predetermined statistical method to the ongoing therapeutic alliance status data.

Example 24 includes a method for clinical outcome predictive analysis, the method comprising: extracting, by a computing device, a plurality of provider features from an assessment instrument, wherein the provider features are indicative of relational characteristics of a care provider; determining, by the computing device, a clinical outcome performance metric associated with the care provider; linking, by the computing device, the plurality of provider features and the clinical outcome performance metric in training data; training, by the computing device, a prediction model with the training data to predict an outcome score based on provider feature, wherein the outcome score is indicative of the clinical outcome performance metric; and determining, by the computing device, a normalization factor for the predicted outcome performance metric to a predicted outcome index.

Example 25 includes the subject matter of Example 24, and wherein the assessment instrument integrates a plurality of validated psychometric instruments.

Example 26 includes the subject matter of any of Examples 24 and 25, and wherein the relational characteristics are indicative of attachment style, self-efficacy, and personality characteristics of the care provider.

Example 27 includes the subject matter of any of Examples 24-26, and wherein the plurality of provider features comprises a plurality of composite or aggregate scores that are each associated with a domain.

Example 28 includes the subject matter of Example 27, and wherein the clinical outcome performance metric comprises a composite z-score based on the plurality of composite or aggregate scores.

Example 29 includes the subject matter of any of Examples 24-28, and wherein determining the clinical outcome performance metric comprises retrieving the clinical outcome performance metric from an organization electronic health records system.

Example 30 includes the subject matter of Example 29, and wherein the clinical outcome performance metric is indicative of a predetermined key performance indicator.

Example 31 includes the subject matter of any of Examples 24-30, and wherein the clinical outcome performance metric is indicative of patient engagement, treatment completion, and discharge against medical advice.

Example 32 includes the subject matter of any of Examples 24-31, and wherein training the prediction model comprises training a machine learning regression model.

Example 33 includes the subject matter of any of Examples 24-32, and further comprising: extracting, by the computing device, a second plurality of provider features from the assessment instrument, wherein the second provider features are indicative of relational characteristics of a second care provider; predicting, by the computing device, a predicted outcome score based on the second plurality of provider features using the prediction model in response to training the prediction mode; and normalizing, by the computing device, the predicted outcome score to a first predicted outcome index as a function of the normalization factor.

Example 34 includes a method for clinical outcome predictive analysis, the method comprising: extracting, by a computing device, a plurality of provider features from an assessment instrument, wherein the provider features are indicative of relational characteristics of a first care provider; predicting, by the computing device, a predicted outcome score based on the plurality of provider features using a pre-trained prediction model, wherein the predicted outcome score is indicative of a clinical outcome performance metric, and wherein the prediction model is trained to predict a predicted outcome score based on provider features; and normalizing, by the computing device, the predicted outcome score to a first predicted outcome index.

Example 35 includes the subject matter of Example 34, and wherein the assessment instrument integrates a plurality of validated psychometric instruments.

Example 36 includes the subject matter of any of Examples 34 and 35, and wherein the relational characteristics are indicative of attachment style, self-efficacy, and personality characteristics of the care provider.

Example 37 includes the subject matter of any of Examples 34-36, and wherein the plurality of provider features comprises a plurality of composite or aggregate scores that are each associated with a domain.

Example 38 includes the subject matter of Example 37, and wherein the clinical outcome performance metric comprises a composite z-score based on the plurality of composite or aggregate scores.

Example 39 includes the subject matter of any of Examples 34-38, and wherein normalizing the predicted outcome score comprises normalizing the predicted outcome score as a function of a predetermined normalization factor.

Example 40 includes the subject matter of any of Examples 34-39, and wherein the pre-trained prediction model comprises a machine learning regression model.

Example 41 includes the subject matter of any of Examples 34-40, and further comprising pairing, by the computing device, a first client to the first care provider based on the first predicted outcome index.

Example 42 includes the subject matter of Example 41, and further comprising: monitoring, by the computing device, for ongoing therapeutic alliance status data associated with the first client and the first care provider; and predicting, by the computing device, a risk of alliance failure based on the ongoing therapeutic alliance status data using a second pretrained prediction model.

Example 43 includes the subject matter of Example 42, and wherein monitoring for the ongoing therapeutic alliance status data comprises connecting to an organization electronic health records system.

Example 44 includes the subject matter of any of Examples 42 and 43, and wherein the ongoing therapeutic alliance status data comprises attendance patterns, engagement with treatment plans, symptom changes, or client feedback.

Example 45 includes the subject matter of any of Examples 42-44, and further comprising automatically assessing, by the computing device, whether predicted outcome scores from the trained prediction model maintain a consistent relationship with performance indicators determined based on the ongoing therapeutic alliance status data.

Example 46 includes the subject matter of Example 45, and wherein automatically assessing the trained prediction model compress applying a predetermined statistical method to the ongoing therapeutic alliance status data.

Example 47 includes computing device comprising: a processor; and a memory having stored therein a plurality of instructions that when executed by the processor cause the computing device to perform the method of any of Examples 24-46.

Example 48 includes one or more machine readable storage media comprising a plurality of instructions stored thereon that in response to being executed result in a computing device performing the method of any of Examples 24-46.

Example 49 includes a computing device comprising means for performing the method of any of Examples 24-46.

1. A computing device for clinical outcome predictive analysis, the computing device comprising:
a provider feature processor configured to extract a plurality of provider features from an assessment instrument, wherein the provider features are indicative of relational characteristics of a care provider;
a clinical performance processor configured to determine a clinical outcome performance metric associated with the care provider;
a model trainer configured to (i) link the plurality of provider features and the clinical outcome performance metric in training data and (ii) train a prediction model with the training data to predict an outcome score based on provider feature, wherein the outcome score is indicative of the clinical outcome performance metric; and
a normalizer configured to determine a normalization factor for the predicted outcome performance metric to a predicted outcome index.

2. The computing device of claim 1, wherein the assessment instrument integrates a plurality of validated psychometric instruments.

3. The computing device of claim 1, wherein the relational characteristics are indicative of attachment style, self-efficacy, and personality characteristics of the care provider.

4. The computing device of claim 1, wherein the plurality of provider features comprises a plurality of composite or aggregate scores that are each associated with a domain.

5. The computing device of claim 4, wherein the clinical outcome performance metric comprises a composite z-score based on the plurality of composite or aggregate scores.

6. The computing device of claim 1, wherein to determine the clinical outcome performance metric comprises to retrieve the clinical outcome performance metric from an organization electronic health records system.

7. The computing device of claim 6, wherein the clinical outcome performance metric is indicative of a predetermined key performance indicator.

8. The computing device of claim 1, wherein the clinical outcome performance metric is indicative of patient engagement, treatment completion, and discharge against medical advice.

9. The computing device of claim 1, wherein the prediction model comprises a machine learning regression model.

10. The computing device of claim 1, further comprising a prediction engine, wherein:
the provider feature processor is further configured to extract a second plurality of provider features from the assessment instrument, wherein the second provider features are indicative of relational characteristics of a second care provider;
the prediction engine is configured to predict a predicted outcome score based on the second plurality of provider features using the prediction model in response to training of the prediction mode; and
the normalizer is further configured to normalize the predicted outcome score to a first predicted outcome index as a function of the normalization factor.

11. A method for clinical outcome predictive analysis, the method comprising:
extracting, by a computing device, a plurality of provider features from an assessment instrument, wherein the provider features are indicative of relational characteristics of a care provider;
determining, by the computing device, a clinical outcome performance metric associated with the care provider;
linking, by the computing device, the plurality of provider features and the clinical outcome performance metric in training data;
training, by the computing device, a prediction model with the training data to predict an outcome score based on provider feature, wherein the outcome score is indicative of the clinical outcome performance metric; and
determining, by the computing device, a normalization factor for the predicted outcome performance metric to a predicted outcome index.

12. The method of claim 11, wherein the assessment instrument integrates a plurality of validated psychometric instruments.

13. The method of claim 11, wherein the plurality of provider features comprises a plurality of composite or aggregate scores that are each associated with a domain.

14. The method of claim 11, wherein determining the clinical outcome performance metric comprises retrieving the clinical outcome performance metric from an organization electronic health records system.

15. The method of claim 11, further comprising:
extracting, by the computing device, a second plurality of provider features from the assessment instrument, wherein the second provider features are indicative of relational characteristics of a second care provider;
predicting, by the computing device, a predicted outcome score based on the second plurality of provider features using the prediction model in response to training the prediction mode; and
normalizing, by the computing device, the predicted outcome score to a first predicted outcome index as a function of the normalization factor.

16. A computing device for clinical outcome predictive analysis, the computing device comprising:
a provider feature processor configured to extract a plurality of provider features from an assessment instrument, wherein the provider features are indicative of relational characteristics of a first care provider;
a prediction engine configured to predict a predicted outcome score based on the plurality of provider features with a pre-trained prediction model, wherein the predicted outcome score is indicative of a clinical outcome performance metric, and wherein the prediction model is trained to predict a predicted outcome score based on provider features; and
a normalizer configured to normalize the predicted outcome score to a first predicted outcome index.

17. The computing device of claim 16, wherein to normalize the predicted outcome score comprises to normalize the predicted outcome score as a function of a predetermined normalization factor.

18. The computing device of claim 16, further comprising an alliance monitor configured to pair a first client to the first care provider based on the first predicted outcome index.

19. The computing device of claim 18, wherein the alliance monitor is further configured to:
monitor for ongoing therapeutic alliance status data associated with the first client and the first care provider; and
predict a risk of alliance failure based on the ongoing therapeutic alliance status data using a second pretrained prediction model.

20. The computing device of claim 19, wherein the ongoing therapeutic alliance status data comprises attendance patterns, engagement with treatment plans, symptom changes, or client feedback.
